(19) United States
(12) Patent Application Publication
(10) Pub. No.: US 2021/0153628 A1
(43) Pub. Date: May 27, 2021

(54) DUAL FUNCTIONING DEODORANT AND MOISTURIZER BOTTLE

(71) Applicant: Brandon JORDAN, Warner Robins, GA (US)

(72) Inventor: Brandon JORDAN, Warner Robins, GA (US)

(21) Appl. No.: 17/095,699

(22) Filed: Nov. 11, 2020

Related U.S. Application Data
(60) Provisional application No. 62/939,558, filed on Nov. 22, 2019.

(51) Int. Cl. A45D 40/18; A45D 34/06; A45D 40/06

(52) U.S. Cl. CPC A45D 40/18; A45D 40/06; A45D 34/06

(57) ABSTRACT

The present disclosure is in the field of containers for consumer products. More particularly, systems and methods are provided for joining two containers at their respective ends and enabling one container to turn and cause contents to be extruded from the other container. Embodiments of the invention comprise two containers of personal care products attached with their bottom surfaces joined in back-to-back fashion. The containers are positioned upright with one on top of the other such that the container on the bottom is upside down. The lower container, which may contain lotion or moisturizer, is connected to the upper container which contains stick deodorant movable by an internal spindle and lift. The spindle-lift mechanism allows the stick deodorant to move up and down when the upper and lower containers are rotated about one another.

### FIELD OF THE INVENTION

The present disclosure is in the field of containers for consumer products. More particularly, systems and methods are provided for joining two containers at their respective ends and enabling one container to turn and cause contents to be extruded from the other container.

### BACKGROUND OF THE INVENTION

Consumers seek convenience and ease of access in their use of personal care products, whether at home or while traveling. Particularly with skin care products, persons seek to have all their various care products at easy access. Consumers want the products they use to be within reach and easy to open and use. Too often, a user's deodorant and skin care liquids get lost in different parts of the bathroom or in different bathrooms.

When a person is traveling, the opportunity for confusion is greater, with the risk that the traveler will forget to pack an item or accidentally leave an item in a hotel room, perhaps when in a hurry to leave for a business or social event or to depart for the airport. What is needed are systems and methods to promote greater organization and simplicity in gathering and maintaining skin care and other personal care products.

### SUMMARY

Two containers of personal care products are attached with their bottom surfaces joined in back-to-back fashion. The containers are positioned upright with one on top of the other such that the container on the bottom is upside down. The lower container, which may contain lotion or moisturizer, is connected to the upper container which contains stick deodorant movable by an internal spindle. The lower container functions as a wheel such that turning the lower container while holding the upper container stationary causes the stick deodorant in the upper container to move upward or downward. Likewise, the lower container may be held in place while the upper container is rotated, which still causes the stick deodorant in the upper container to move upward or downward.

### BRIEF DESCRIPTION OF FIGURES

FIG. 1 through FIG. 9 are diagrams of a dual functioning deodorant and moisturizer bottle according to various embodiments of the present disclosure.

### DETAILED DESCRIPTION

Systems and methods described herein provide two containers of personal care products, the bottom ends, also referred to as the proximal ends, of which are connected to each other. The top or dispensing ends, also referred to the distal ends, face away from each other in opposite directions. When in normal upright standing position, one container is on top of the other. The upper container is itself upright with its dispensing end at the top and pointing upward. The lower container is directly below the upper container with its dispensing component facing downward.

The lower container is upside down with its bottom surface pointing upward and attached to the bottom of the upper container. The dispensing end of the lower container, which when upright is at the top and pointing upward, in this case is at the bottom and points downward as noted. The two containers have a divider between them that allows the containers to remain attached and be rotated relative to each other.

In some embodiments, the upper container is stick deodorant or solid deodorant container comprising an inner portion and an outer portion, wherein the fragrant wax-like deodorant substance contained within the inner portion of the container is pushed out by an internal spindle threaded with a lift. In well-known containers of such stick deodorant, a wheel is at the bottom of the container. Turning the wheel turns the spindle, thereby translating the lift along the spindle which slowly forces the deodorant substance up and out of the container for application in the user's underarms.

The present disclosure provides that an attached and upside-down bottle containing lotion or other liquid care product performs the function of the wheel. The bottoms of the two containers are attached in the aforementioned back-to-back fashion. The bottom of the lotion container is attached as a wheel or turning mechanism of the deodorant container.

In some embodiments, a user may grasp the upright deodorant container with one hand and hold it steady. The user may then grasp the downward-pointing lotion container and turn it. Depending on the direction of the turning, this action will cause the deodorant substance to either protrude/extend from or recess/retract back into the deodorant container. As noted, this is because the lotion container is connected to the turning function of the deodorant container and is effectively a turning handle for the deodorant container.

In some embodiments, a user may likewise grasp the downward-pointing lotion container with one hand and hold it steady. The user may then grasp the upward-pointing deodorant container and turn it. These actions will cause the deodorant to protrude/extend from or recess/retract back into the deodorant container depending on the direction of the turning since these actions cause rotation of the spindle, which in turns translates the lift.

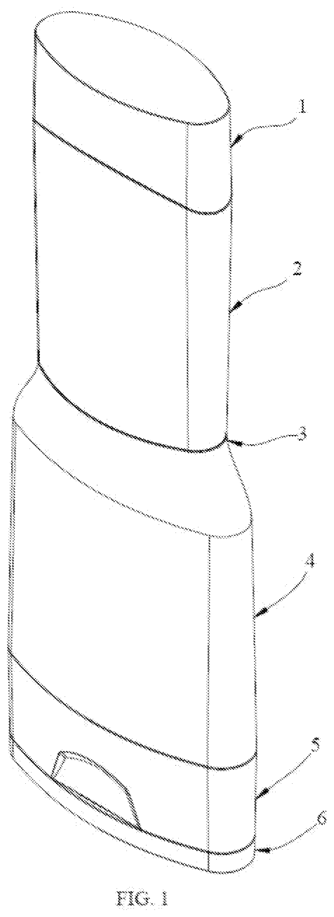

Turning to the figures, FIG. 1 is a diagram of a system of a dual functioning deodorant and moisture bottle according to an embodiment of the present disclosure. Components of the system illustrated in FIG. 1 comprise a deodorant cap 1, a deodorant container 2, a divider 3, a lotion container 4, a lotion cap 5, and a lotion cap top 6. In FIG. 1 and figures to follow, the deodorant container 2 and its associated parts are on the top and upright and the lotion container 4 and its associated parts are on the bottom and upside down. In some embodiments, the divider 3 is the component that provides connection.

In some embodiments, the divider comprises the proximal end of the lotion container. In these embodiments, the divider is fixed to the lotion container in a manner that does not allow the divider to rotate relative to the lotion container. The deodorant container may be connected to the lotion container by means of the divider in a manner that allows the deodorant container to rotate relative to the lotion container, hence also rotate relative to the divider. The spindle may be fixed to the divider in a manner that does not allow the spindle to rotate relative to the divider, hence does not allow the spindle to rotate relative to the lotion container. In these embodiments, the deodorant container may rotate relative to the spindle. The rotation of the deodorant container relative to the spindle, divider, and lotion container or the rotation of the spindle, divider, and lotion container relative to the deodorant container is what causes the extension or retraction of the solid deodorant within the inner portion of the deodorant container.

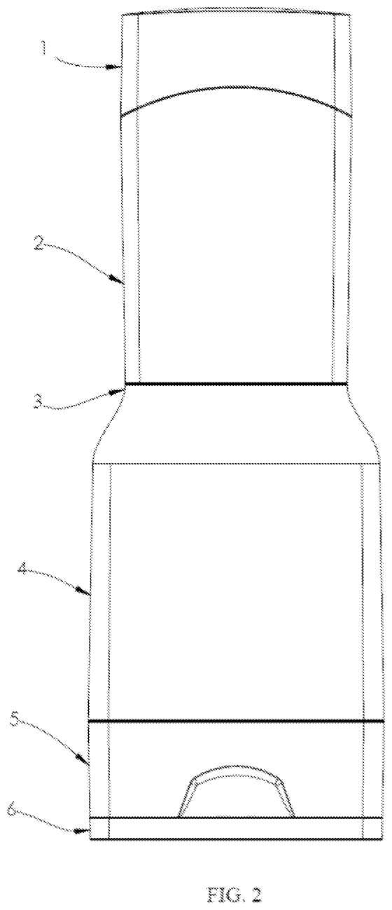

FIG. 2 is a straight frontal view of the system in contrast to the angled view provided in FIG. 1. Component numbering is consistent between all figures provided herein.

The lotion container 4 may be wider than the deodorant container 2, may contain more physical volume that the deodorant container, and may be heavier than the deodorant container. Consequently, the view in FIGS. 1 and 2 is of the default positioning of the system such while the system could be positioned in an opposite manner with the deodorant container 2 on the bottom, the system would be unstable and more easily tip over.

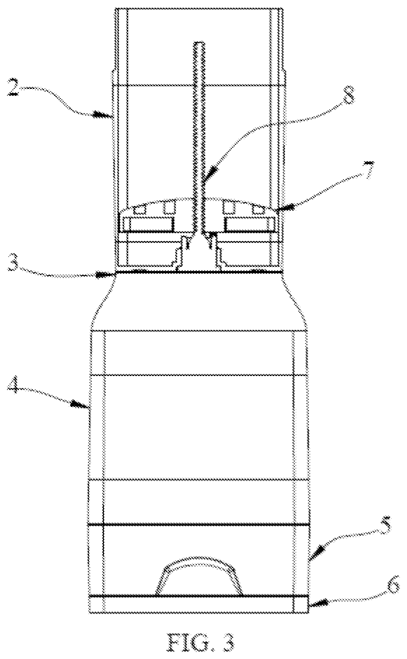

FIG. 3 is a straight frontal view of the system as in FIG. 2 but with some internal components shown. A deodorant lift 7 is shown and a threaded deodorant spindle 8 is also shown in FIG. 3. The deodorant lift 7 is positioned below the deodorant substance and supports the substance as it is being moved up or down. The threaded deodorant spindle 8 turns, maintains contact with the deodorant lift 7, and propels the deodorant lift 7 up or down. In some embodiments, the lift 7 may be threaded onto the threaded spindle 8 so that when the threaded spindle 8 rotates, the lift 7 is translated along the threaded spindle 8.

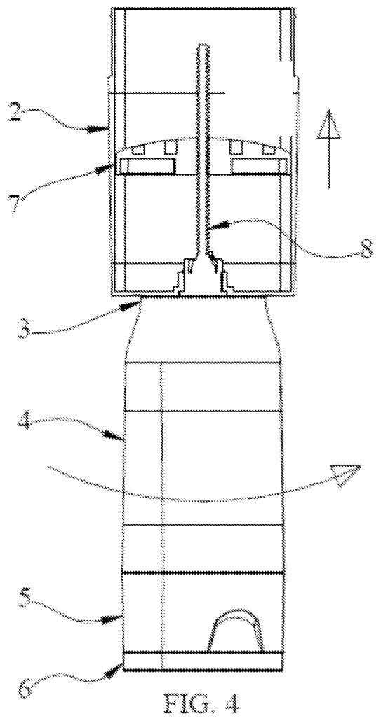

FIG. 4 depicts the deodorant container 2 being turned with arrows in each figure. In FIG. 4 the lotion container 4 is moving as indicated by the arrow and the deodorant container 2 is stationary. The lotion container 4 is depicted at an angle and not facing straight on to the viewer as the deodorant container 2 is doing in FIG. 4.

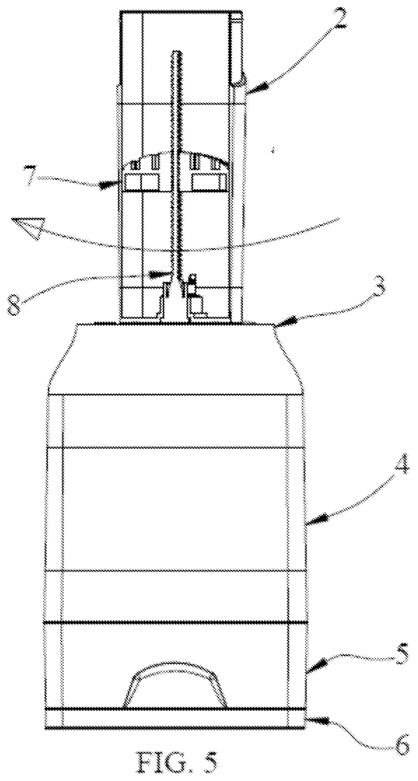

In contrast to FIG. 4, FIG. 5 conversely depicts the deodorant container 2 being turned and the lotion container 4 as stationary, hence the angled view of the deodorant container 2. The components in FIG. 4 and FIG. 5 appear to be of different sizes because some of the components are in the process of turning. The components actually are of the same sizes as in FIGS. 1-3.

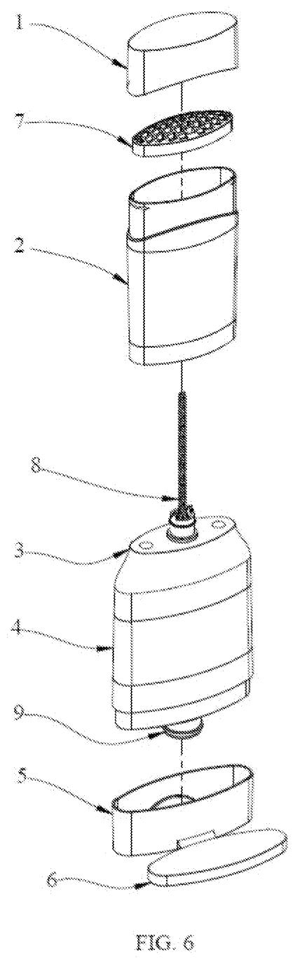

FIG. 6 is a linear exploded view of the components. A lotion hole 9 is shown allowing lotion to move from the lotion container 4 to the lotion cap 5.

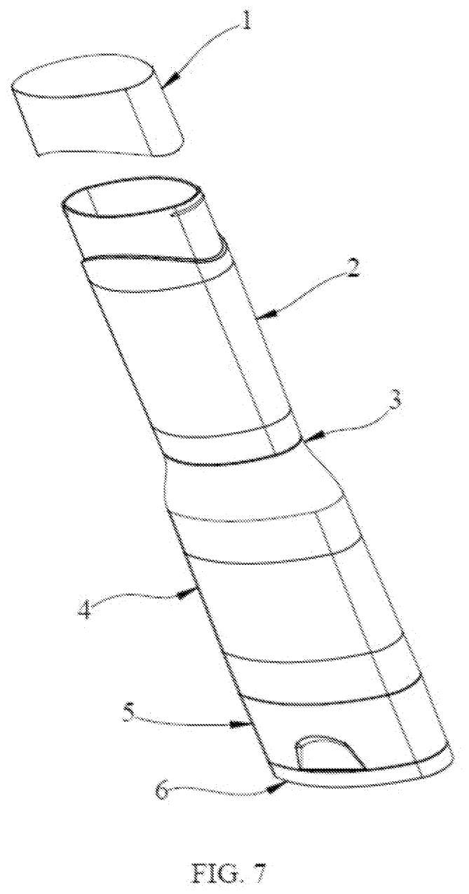

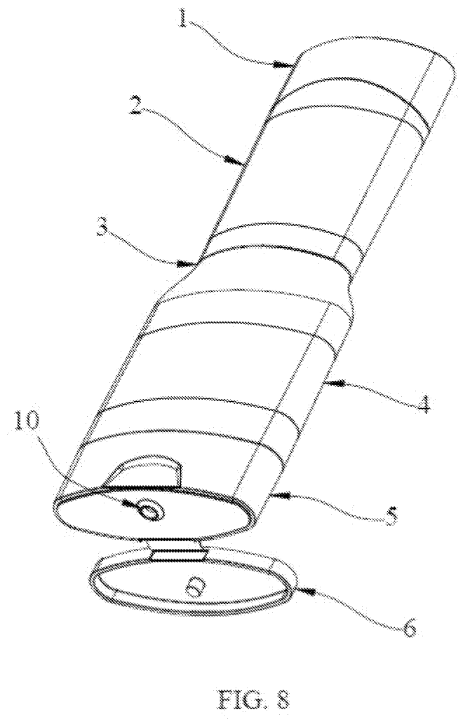

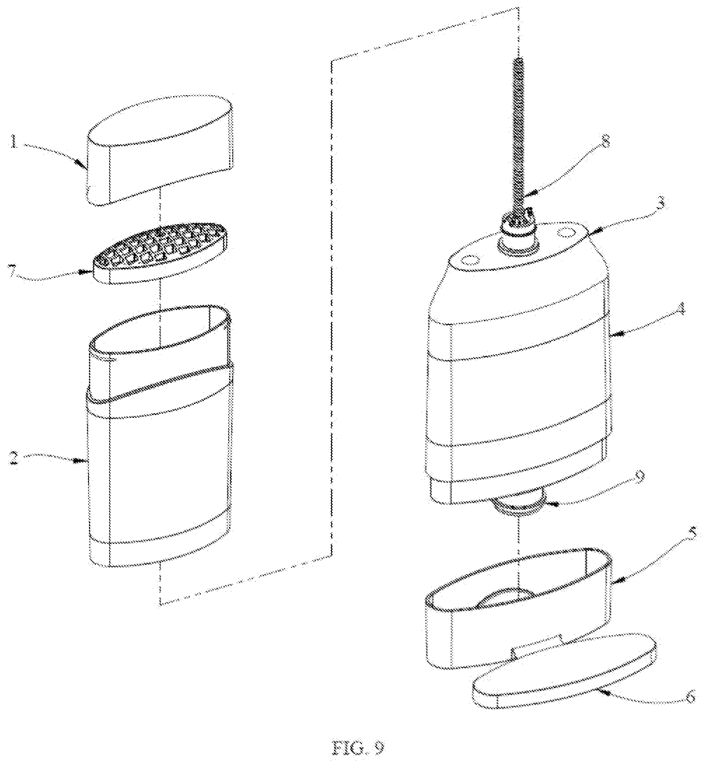

FIG. 7 is a view of the system in closed state. An exception is the deodorant cap 1 which is shown in detached state. Similar to FIG. 7, FIG. 8 is a view of the system in closed state. An exception in FIG. 8 is the open state of the lotion cap top 6 which reveals the lotion dispenser hole 10. FIG. 9 is a another fully exploded view of the system with all components shown except the lotion dispenser hole 10.

While discussion herein has described the lotion container 4 as containing lotion or moisturizer, in embodiments the lotion container 4 may contain other substances. For example, the lotion container could contain shampoo, shaving solution, after-shave liquid, or any other liquid that serves as a skincare or personal care product. Furthermore, the solid deodorant container my contain any variety of solid deodorant, including antiperspirant. The solid deodorant is described herein as being fragrant and wax-like. However, this is not to be interpreted as limiting the solid deodorant to having a fragrance or having physical or chemical properties similar to those of wax.

In some embodiments, the lotion container 4 would need to be squeezed by the user to cause contents to be dispensed. The deodorant container 2 also can be rotated to allow the deodorant to move up and down.

What is claimed is:

1. a dual container system comprising:
a liquid container containing liquid, the liquid container comprising a distal end and a proximal end;
a threaded spindle attached to the liquid container at the proximal end;
a lift threaded onto the threaded spindle;
a deodorant container containing solid deodorant, the deodorant container comprising a distal end, a proximal end, an inner portion, and an outer portion, the proximal end of the deodorant container being connected to the proximal end of the liquid container so that the spindle and lift are disposed within the inner portion of the deodorant container, wherein the lift contacts the solid deodorant so that when the liquid container and deodorant container are rotated relative to one another the lift is translated along the threaded spindle thereby extending or retracting the solid deodorant from or into the deodorant container through the distal end of the deodorant container.

2. the dual container system of claim 1, wherein the distal ends of both the deodorant container and the liquid container comprise removable caps.

3. the dual container system of claim 1, wherein the removable cap of the liquid container further comprises a liquid container top.

4. the dual container system of claim 1, wherein the liquid is a skincare product.

5. the dual container system of claim 1, wherein the solid deodorant is an antiperspirant.

6. the dual container system of claim 1, wherein the liquid and the solid deodorant are separated by a divider.

7. the dual container system of claim 6, wherein the proximal ends of the deodorant container and the liquid container are connected using the divider.

8. the dual container system of claim 7, wherein the divider comprises the proximal end of the liquid container.

9. the dual container system of claim 1, wherein the distal end of the liquid container serves as a base of the dual container system.

10. the dual container system of claim 9, wherein the distal end of the liquid container serves as the bottom of the dual container system and wherein the distal end of the deodorant container serves as the top of the dual container system.

11. A method for joining two containers, comprising;
attaching a threaded spindle to a proximal end of a first container; and
attaching a proximal end of a second container to the proximal end of the first container so that the threaded spindle is disposed within an inner portion of the second container.

12. the method of claim 11, wherein the first container is a liquid container containing liquid.

13. the method of claim 11, wherein the second container is a deodorant container containing solid deodorant.

14. he method of claim 12, wherein the liquid is a skincare product.

15. the method of claim 11, further comprising separating the first container and second container by use of a divider.

16. The method of claim 15, wherein upon implementation of the divider, the divider comprises the proximal end of the first container.

17. the method of claim 11, further comprising storing the joined containers by standing the containers one on top of another to create a stack of two joined containers, wherein the distal end of the first container serves as the bottom of the stack of two joined containers, and wherein the distal end of the second container serves as the top of the stack of two joined containers.
